(19) United States
(12) Patent Application Publication
(10) Pub. No.: US 2005/0008539 A1
(43) Pub. Date: Jan. 13, 2005

(54) Sensor informationlinkage apparatus

(76) Inventors: Satoshi Matsuura, Kyoto (JP); Atsushi Nakamura, Kanagawa (JP); Atsushi Hattori, Tokyo (JP); Tomohiro Inagawa, Kanagawa (JP); Takashi Kashimoto, Nara (JP); Keiji Ogawa, Nara (JP)

Correspondence Address: HARNESS, DICKEY & PIERCE, P.L.C., P.O. BOX 828, BLOOMFIELD HILLS, MI, 48303, US

(21) Appl. No.: 10/498,413

(22) PCT Filed: Dec. 16, 2002
(86) PCT No.: PCT/JP02/13118

(30) Foreign Application Priority Data
Dec. 14, 2001 (JP) 2001-382247

(51) Int. Cl. G01N 33/00

(52) U.S. Cl. 422/68.1; 700/12

(57) ABSTRACT

For the purpose of associating a device having a sensor with a device which utilizes a detection result so as to enable various operation control, a refrigerator includes a temperature sensor for detecting a compartment temperature, a storage section for storing a detection result, an information association control section for controlling transmission of the detection result to a microwave oven, and the like, and an NIC for communicating with the microwave oven through a network, or the like. The microwave oven includes an NIC of the same type, an information association control section for controlling reception of the compartment temperature transmitted from the refrigerator, and the like, and a principal part control section for controlling the operation of principal part of the microwave oven. The microwave oven heats food taken out of the refrigerator by performing heating control according to the compartment temperature at the time when the food is taken out of the refrigerator, i.e., the core temperature of the food.

### TECHNICAL FIELD

The present invention relates to a device which performs control based on detection with a sensor, such as a heating cooking appliance in which the heating power is controlled according to the temperature of a material to be cooked, a cloth dryer in which the degree of heating is controlled according to the ambient temperature. Specifically, the present invention relates to a sensor information association device capable of effectively using a detection result obtained by a sensor among a plurality of devices.

### BACKGROUND ART

For example, in many household electric appliances, or the like, the operation of the appliance is controlled according to the outputs of various sensors. Specifically, in a microwave oven, the temperature at the surface of a material to be cooked is detected to control the heating power. In the case of a cloth dryer, the temperature inside the dryer is detected to control the degree of heating.

However, in some cases, a detection target for performing optimum control is not necessarily readily detected. For example, in the example of the microwave oven, if not only the temperature at the surface of a material but also the temperature of the inner part of the material (core temperature) is detected, it is possible to uniformly heat the material such that the inner part of the material is not left cold. In the case of a cloth dryer, it is possible to control the dried state based on not only the temperature inside the dryer but also the ambient temperature. However, it is not realistic when the arrangement of wires, or the like, is considered.

For example, in an example described in Japanese Unexamined Patent Publication No. 2001-133100, dispersedly provided controllers in a store management system transmit/receive the temperature detected by a temperature/humidity sensor. The controllers collectively constitute a store management system although they are dispersedly provided. In the entire system, a necessary number of sensors are provided at necessary places. That is, even if the detected temperature is transmitted among the controllers, it is impossible to make what cannot be detected by the system detectable.

### DISCLOSURE OF INVENTION

In view of the above, an objective of the present invention is to readily obtain a detection result for a detection target which is difficult to detect with a single device, such that the operation control of the device is performed more appropriately, and various operation control are performed.

In order to achieve the above objective, the first sensor information association device of the present invention comprises: a sensor; detection result request reception means for receiving from another device a request for a detection result of the sensor, the another device being capable of operating independently; and detection result transmission means for transmitting the detection result to the another device according to the request.

With the above structure, even when the another device does not have such a sensor, the detection result of the sensor is transmitted to the another device, and the operation of the another device is controlled based on the detection result, whereby more appropriate operation control and a broader variety of operation control are achieved as compared with a case where the another device operates independently.

The second sensor information association device of the present invention is based on the first sensor information association device, which further comprises storage means for storing the detection result at a predetermined timing determined according to the status of the sensor information association device, wherein the detection result transmission means transmits the detection result stored in the storage means.

The third sensor information association device of the present invention is based on the second sensor information association device, wherein: the sensor information association device and the another device deal with a same material; and the predetermined timing is a timing determined according to an user operation of transferring the material to the sensor information association device.

With the above structures, even if the power of the device is not on at a time when a detection result which is necessary for another device to control its operation is obtained, the detection result can be provided when the operation control is actually performed. Specifically, in the case of thawing food taken out of the freezer compartment by a microwave oven, even if the compartment temperature is detected at a time when the food is taken out of the freezer compartment while the power of the microwave oven is off, the compartment temperature is utilized when the food is heated in the microwave oven such that a desirable heating operation is performed.

The fourth sensor information association device is based on the first sensor information association device, which further comprises search means for searching for the another device which is capable of requesting the detection result, wherein the detection result transmission means transmits the detection result according to the another device found by the search means.

The fifth sensor information association device is based on the fourth sensor information association device, which further comprises a provided sensor information database in which information indicating a data item detected by the sensor is registered, wherein the search means searches for a device in which the data item registered in the provided sensor information database is usable.

The sixth sensor information association device is based on the fourth sensor information association device, which further comprises an associated device database in which information indicating candidates of a device which uses the detection result is registered, wherein the search means searches for a device capable of communicating the detection result from among the candidate devices registered in the associated device database.

With the above structures, a device to which the detection result can be provided and a device in which the detection result is usable are automatically associated with each other.

The seventh sensor information association device is a sensor information association device capable of operating independently, comprising: detection result request means for requesting a detection result obtained by a sensor provided to another device; and detection result reception means for receiving the detection result transmitted in response to the request.

With the above structure, even a detection result which is obtained by a sensor that is not included in the sensor information association device is acquired from the another device having such a sensor, and operation control is performed based on the acquired detection result, such that appropriate operation control and a variety of operation control are achieved.

The eighth sensor information association device is based on the seventh sensor information association device, wherein the detection result request means requests the detection result at a predetermined timing determined according to the status of the sensor information association device.

The ninth sensor information association device is based on the eighth sensor information association device, wherein the predetermined timing is a timing when a user performs an operation of instructing the sensor information association device to perform operation control using the detection result obtained by the sensor.

The tenth sensor information association device is based on the eighth sensor information association device, wherein the predetermined timing is a timing when a user performs an operation which is expected to instruct the sensor information association device to perform operation control using the detection result obtained by the sensor.

With the above structures, even if the power of the sensor information association device is off at a time when a detection result is obtained by a device having a sensor, the detection result can be obtained at a timing when the sensor information association device performs operation control. Furthermore, in the case where the detection result of the sensor is not utilized, the load of transmitting the detection result in vain is reduced.

The eleventh sensor information association device is based on the seventh sensor information association device, which further comprises search means for searching for a device to which the detection result can be transmitted, wherein the detection result request means requests the detection result from the device found by the search means.

The twelfth sensor information association device is based on the eleventh sensor information association device, wherein the search means searches for a device to which the detection result can be transmitted prior to a determination that the detection result obtained by the sensor is used.

The thirteenth sensor information association device is based on the eleventh sensor information association device, wherein when a possibility of using the detection result occurs, the search means searches for a device to which the detection result can be transmitted.

The fourteenth sensor information association device is based on the eleventh sensor information association device, which further comprises a request sensor information database in which information indicating a data item detected by the sensor and usable for the sensor information association device is registered, wherein the search means searches for a device to which the data item registered in the request sensor information database can be provided.

The fifteenth sensor information association device is based on the eleventh sensor information association device, which further comprises an associated device database in which information indicating candidates of a device to be provided with the detection result is registered, wherein the search means searches for a device capable of communicating the detection result from among the candidate devices registered in the associated device database.

With the above structures, a device to which the detection result can be provided and a device in which the detection result is usable are automatically associated with each other as described about the fourth device.

The sixteenth sensor information association device is based on the first sensor information association device, wherein the sensor is a temperature sensor.

The seventeenth sensor information association device is based on the sixteenth sensor information association device, wherein: the sensor information association device is a refrigerator; the another device is a heating cooking appliance; and the sensor is a temperature sensor for detecting a compartment temperature of the refrigerator.

The eighteenth sensor information association device is based on the seventh sensor information association device, wherein the sensor is a temperature sensor.

The nineteenth sensor information association device is based on the eighteenth sensor information association device, wherein: the sensor information association device is a heating cooking appliance; the another device is a refrigerator; the sensor is a temperature sensor for detecting a compartment temperature of the refrigerator; and the heating cooking appliance performs heating control based on the received detection result.

With the above structures, even a microwave oven which does not have a temperature sensor can perform operation control based on the detected temperature. The core temperature of a material to be heated, which is difficult to detect in a heating device, such as a microwave oven, or the like, is acquired by obtaining from a refrigerator, or the like, the ambient temperature (compartment temperature) of a place where the material was stored, and heating control is performed according to the acquired core temperature such that appropriate thawing and heating are achieved.

The microwave oven and the refrigerator operate independently from each other and do not simultaneously work on the same material to perform a process, such as cooking, or the like. Thus, these appliances carry out their functions independently. However, by transmitting the above-described sensor information, the microwave oven is enabled to perform a more appropriate heating process, or the like.

### BRIEF DESCRIPTION OF DRAWINGS

FIG. 1 is a block diagram showing the structure of principal part of a refrigerator and a microwave oven according to embodiment 1 of the present invention.

FIG. 2(a) illustrates an example of a provided information definition which is stored in a refrigerator 101 according to embodiment 1.

FIG. 2(b) illustrates an example of a request information definition which is stored in a microwave oven 201 according to embodiment 1.

FIG. 3 is a flowchart illustrating the operation of the refrigerator and the microwave oven according to embodiment 1.

FIG. 4 is a flowchart illustrating the operation of the refrigerator and the microwave oven according to embodiment 2 of the present invention.

FIG. 5 illustrates an example of a sensor according to another embodiment.

### BEST MODE FOR CARRYING OUT THE INVENTION

(Embodiment 1)

Hereinafter, an example of embodiment 1 of the present invention is described, wherein a refrigerator and a microwave oven, which are sensor information association devices, are associated with each other, and a detection result of the temperature inside the refrigerator is utilized such that the microwave oven performs an appropriate heating operation.

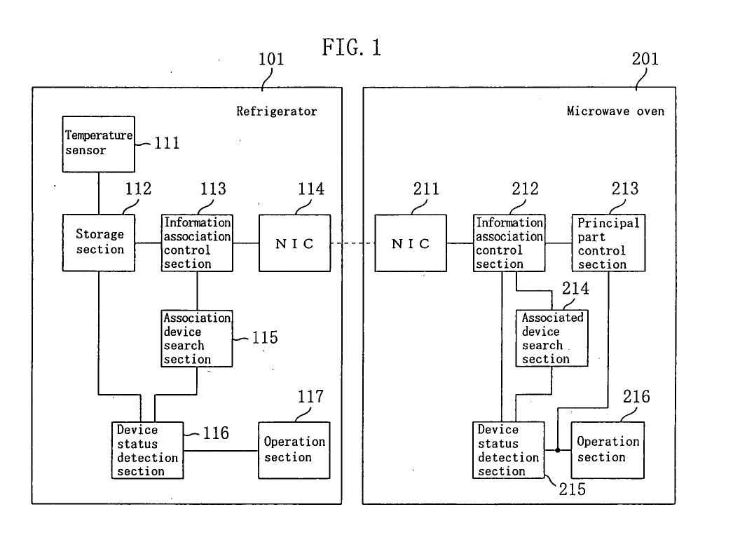

FIG. 1 is a block diagram showing the structure of principal part of a refrigerator 101 and a microwave oven 201 according to embodiment 1 of the present invention.

In FIG. 1, a temperature sensor 111 detects the compartment temperature of the refrigerator 101. In the case where the refrigerator 101 has a freezer compartment, a chilled compartment, and the like, the temperature sensor 111 is preferably provided to each compartment.

A storage section 112 stores a detection result obtained by the temperature sensor 111. For example, at the time when a material to be cooked is taken out of the refrigerator 101, the microwave oven 201 is not necessarily in an operating state (power on). Thus, in such a case, the storage section 112 stores the compartment temperature detected at the time when the material to be cooked is taken out.

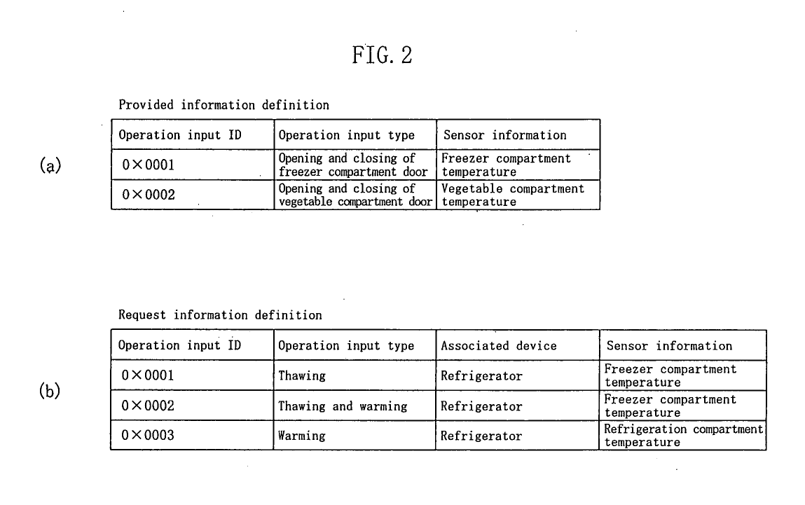

An information association control section 113 controls, for example, transmission of a detection result of the compartment temperature detected by the temperature sensor 111 such that the result is available at the microwave oven 201. The information association control section 113 stores a provided information definition (provided sensor information database) which shows information that can be provided by the refrigerator 101. Specifically, for example, as shown in FIG. 2(a), the information association control section 113 stores the data items detected by the sensor and information showing the statuses of the refrigerator 101 (for example, the operations performed by a user) in which providable results were detected. It should be noted that the user operation for which the detected result can be provided is not limited to a single user operation but may be a series of user operations. The information stored in the information association control section 113 is not limited to the above information. In the case where the information which is provided in conjunction with an associated device is determined, the information showing a device that can be associated with (association device database) may be stored in the information association control section 113.

An NIC (Network Interface Card) 114 establishes a communication with the microwave oven 201 through a wired or wireless network, or the like.

An association device search section 115 searches for an association device (e.g., the microwave oven 201) which is connected to the refrigerator 101 through a network, or the like, and in which the compartment temperature of the refrigerator 101 can be used for control, or the like.

A device status detection section 116 detects the status of the refrigerator 101 according to, for example, opening and closing of the door by a user, an operation performed by a user with a console panel, or the like.

An operation section 117 outputs an operation input ID which represents the user operation performed at the door of the refrigerator 101, a console panel for setting the compartment temperature of the refrigerator 101, or the like. The operation input ID is input to a control section of the principal part of the refrigerator (not shown) and the device status detection section 116 or to the device status detection section 116 through the control section.

An NIC 211 of the microwave oven 201 establishes a communication with the refrigerator 101 through a network as does the NIC 114.

An information association control section 212 controls, for example, reception of the compartment temperature transmitted from the refrigerator 101. The information association control section 212 stores a request information definition (request sensor information database) which shows information (usable information) requested by the microwave oven 201. Specifically, for example, as shown in FIG. 2(b), the information association control section 212 stores requested data items of the sensor and information showing the statuses of the microwave oven 201 (for example, the operations performed by a user) in which the detection result is necessary. The information stored in the information association control section 212 is not limited to the above information. In the case where the requested information is determined according to an associated device, the information indicating a candidate of a device that can be associated with (association device database) may be stored in the information association control section 212, as described in the refrigerator 101. Furthermore, information that shows in which status of a partner device the detection result is necessary, or the like, may be stored.

A principal part control section 213 controls the operation of the principal part of the microwave oven, e.g., controls the heating power.

An associated device search section 214 searches for an associated device (e.g., the refrigerator 101) which is connected to the microwave oven 201 through a network, or the like, and which can provide a detection result usable in a control operation, or the like, in the microwave oven 201.

A device status detection section 215 detects the status of the microwave oven 201 according to, for example, opening and closing of the door by a user, an operation performed by a user with a console panel, or the like.

An operation section 216 outputs an operation input ID which represents the user operation performed at the door of the microwave oven 201, a console panel for setting the heating temperature, or the like. The operation input ID is input to the principal part control section 213 and the device status detection section 116 or to the device status detection section 116 through the control section.

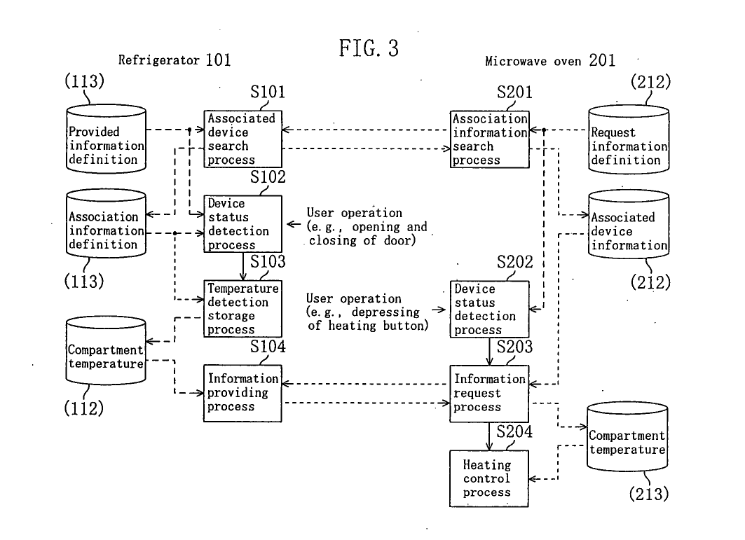

Next, the operation of the refrigerator 101 and the microwave oven 201 which have the above structures is described with reference to the flowchart of FIG. 3.

In the first place, each or any one of the refrigerator 101 and the microwave oven 201 searches for, at a predetermined timing, an associated device which can provide a detection result of a sensor or in which a detection result of a sensor is usable (S101, S201). In many cases, the predetermined timing is preferably asked by the microwave oven 201 because the power of the refrigerator 101 is on all the time whereas the microwave oven 201 is powered on only when it is used, but the present invention is not limited thereto. For example, the timing may be the time when the device is used for the first time (when powered on or when connected to a network), the time when passage of a predetermined time period is detected, or the time when a search instruction operation is performed by a user. (Alternatively, the above search may be conducted when the door of the microwave oven 201 is opened or when a heating instruction operation is performed, as will be described in embodiment 2.)

More specifically, the search for the associated device is conducted by mutually referring to the data items detected by a sensor which are stored in the information association control sections 113 and 212 of the refrigerator 101 and the microwave oven 201 and which can be provided by or usable in each of these device. Thus, in the example of embodiment 1, the refrigerator 101 finds the microwave oven 201 as a device in which the compartment temperature of the refrigerator 101 is usable, while the microwave oven 201 finds the refrigerator 101 as a device which can provide the compartment temperature. The search results are stored as associated device information in the information association control sections 113 and 212.

Thereafter, if the status shown in the provided information definition (FIG. 2(a)), e.g., opening and closing of the door of the refrigerator 101, is detected by the device status detection section 116 of the refrigerator 101 (S102), the compartment temperature at this time, which corresponds to the opened and closed door, is detected by the temperature sensor 111 and stored in the storage section 112 (S103). The search results are once stored because the microwave oven 201 can be notified later about the results after it is powered on even if the power of the microwave oven 201 is off at the time when the search results are obtained. The results may be immediately notified when the power of the microwave oven 201 is on. Alternatively, a variation in the operation state of a compressor or the count value of a timer is detected separately from the timing of opening and closing the door, and the history of the compartment temperature is stored, such that the reliability of the detected temperature can be determined according to whether or not the compartment temperature is in a stationary state or is varying.

Then, if the status shown in the request information definition (FIG. 2(b)), e.g., powering on, opening and closing of the door, a temperature setting operation, a heating start operation, or the like, is detected by the device status detection section 215 of the microwave oven 201 (S202), the information association control section 212 of the microwave oven 201 requests from the refrigerator 101 the compartment temperature previously detected at the time when the door is opened and closed (S203). In response to the request, the information association control section 113 of the refrigerator 101 notifies the microwave oven 201 about the compartment temperature stored in the storage section 112 (S104). Herein, if the door of the refrigerator 101 is opened and closed a plurality of times within a predetermined time period, the compartment temperature detected at each point in time may be notified, or only the compartment temperature detected at the time of immediately previous opening and closing may be notified. Alternatively, the variation in the compartment temperature which is caused by opening and closing of the door may be corrected before notification. (It should be noted herein that even if the compartment temperature is varied due to opening and closing of the door, the temperature of the content of the refrigerator does not immediately change.) Further, the time of opening and closing of the door or the time elapsed after that may be notified together with the compartment temperature. For example, the time period between the time when a material to be cooked is taken out of the refrigerator 101 and the time when it is cooked in the microwave oven 201 is long, the influence of an increase in temperature due to room temperature is considered according to the time period.

The principal part control section 213 of the microwave oven 201 controls the heating power or heating power distribution for a material to be cooked, a method for heating the material, or the like, based on the notified temperature, a heating instruction operation by a user, and the like (S204). For example, if the material to be cooked is kept in the refrigerator 101 for a certain time period, the temperature of the material is equal to the compartment temperature. Thus, the compartment temperature is obtained, and heating control is performed according to the temperature, especially the core temperature, of the material, whereby an appropriate heating operation is readily performed. For example, in the case of thawing frozen Sashimi (slices of raw fish) which is generally difficult to thaw, the Sashimi is readily and sufficiently thawed while being kept raw. It should be noted that even if the compartment temperature is notified by the refrigerator 101 as described above, it is not necessary to perform control according to the compartment temperature. For example, in the case where an instruction to simply warm a material is made at the microwave oven 201 while no thawing instruction is made although the door of a freezer compartment is opened and closed, or in the case where it is determined based on the detection with a surface temperature sensor of the microwave oven 201 (even if the elapse of time is considered) that there is a scarce possibility of heating frozen food in the microwave oven 201, heating control is performed under other generally-employed conditions irrespective of the freezer compartment temperature. Alternatively, in the case where the doors of both the freezer compartment and the chilled compartment of the refrigerator 101, which have different compartment temperatures, are opened and closed, it is determined by detection with the surface temperature sensor from which compartment a material placed inside the microwave oven 201 has been taken out. Further, the variation in temperature of a material to be cooked after the material is taken out of the refrigerator 101, the temperature distribution in the material, or the like, may be estimated according to the time elapsed after the door of the refrigerator 101 was opened and closed, the surface temperature of a material to be cooked, the heat capacity of the material, the room temperature (e.g., obtained from an air conditioner), or the like, in addition to the notified information, and heating control may be performed according to the estimated variation or distribution.

Although in the above example the refrigerator 101 notifies the microwave oven 201 about the temperature, the microwave oven 201 may notify the refrigerator 101 about the temperature. For example, now consider a case where the microwave oven 201 can determine cooking details (e.g., in the case where the microwave oven 201 has the function of receiving a designated cooking menu or the function of displaying a cooking procedure). In the case of making pudding wherein a pudding material is heated in the microwave oven 201 and then cooled in the refrigerator 101, the refrigerator 101 is notified about the heating temperature in the microwave oven 201, or the like, whereby the operation of the refrigerator 101 is determined before an increase in the compartment temperature is detected, e.g., the pudding material is quickly cooled or the cooling temperature is controlled according to the heating temperature. Further, it may be announced according to the variation in the compartment temperature or passage of time during cooling of the pudding that the pudding is cooled to a temperature appropriate for eating. The microwave oven 201 may be notified about the compartment temperature and announce completion of cooking according to the notified temperature.

The notification is not limited to a notification transmitted between the refrigerator 101 and the microwave oven 201. For example, a notification of temperature, humidity, or the like, may be transmitted between a cloth dryer and an air conditioner. In the case where clothes are dried by both a dryer and natural air drying, the time required the natural air drying is determined according to the degree of drying by the dryer and the temperature and humidity. The temperature and humidity inside and outside the room are detected by sensors provided to an indoor unit and outdoor unit of the air conditioner and transmitted to the cloth dryer, whereby clothes are surely dried in a desirable natural air drying time. Further, excessive heating is prevented, and accordingly, wear and tear of clothes are prevented, and the electric power is saved. Likewise, the temperature control of a water heater is conducted according to the atmospheric temperature (e.g., the selected temperature is corrected), whereby a desirable sensible temperature is obtained according to the atmospheric temperature.

The target of the sensor is not limited to the temperature and humidity. For example, a sensor provided to each device is used or diverted, such that sound is detected by a microphone of a telephone or interphone, or light is detected by an infrared sensor of a remote control. A sensor which is provided to a device in consideration of a place where the device is installed, or the like factor, but which is not necessary to the control of the device, may be utilized. Such sensors can be variously combined with devices capable of analyzing detection results of the sensors and utilizing the analyzed detection results in a control process.

The detection result of a sensor may not be a detected value itself. The detected value may be processed before being transmitted. For example, the detected value may be averaged or ranked or may be combined with a detection result of a different sensor, or the like, for calculation.

The detection result is not limited to one that is directly transmitted from a device having a sensor to a device which uses the detection result. The detection result may be transmitted through, for example, a device whose power is on all the time, an information management unit for centralizedly managing detection results of various sensors, a personal computer, or the like.

(Embodiment 2)

In an example described herein, when a user performs any operation at the microwave oven 201, a device with which a detection result of a sensor is associated is searched for. In embodiment 2, the structure of the refrigerator 101 and the microwave oven 201 is basically the same as that of embodiment 1 (FIG. 1) except for a control operation performed by the information association control section 113 and the information association control section 212. Further, the request information definition stored in the information association control section 212 of the microwave oven 201 includes, in addition to the information shown in FIG. 2(b), information that indicates the timing of searching for an associable device, e.g., information indicating that the search is performed when the door of the microwave oven 201 is opened.

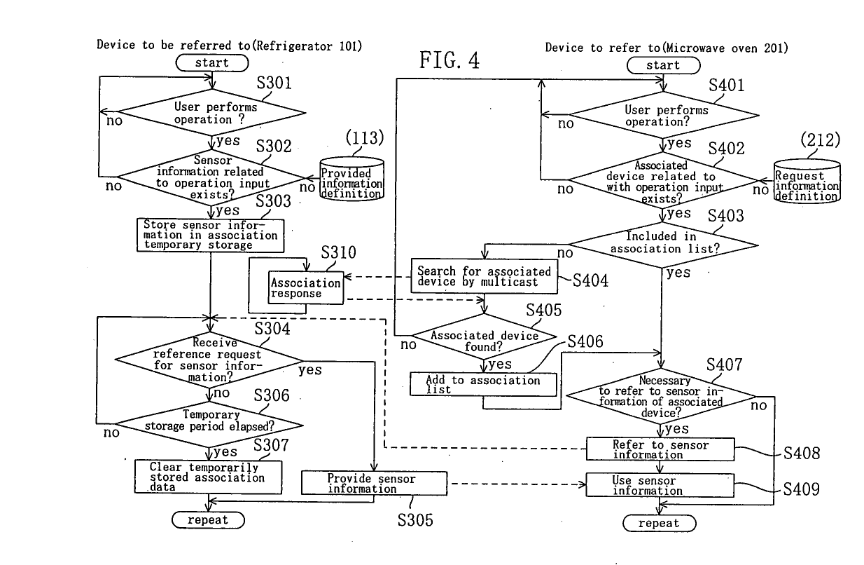

As shown in FIG. 4, the refrigerator 101 determines whether or not a user has performed an operation at the operation section 117 (S301). This determination is repeated till any user operation is detected. When a user operation is detected, it is determined whether or not sensor information related to the user operation, i.e., information indicating the data items detectable at the timing of the user operation, or information indicating some of the data items which can be used in another device, is stored in the provided information definition of the information association control section 113 (S302). If the sensor information is not stored, the above determinations of steps S301 and S302 are repeated. If the sensor information is stored, a detection result of a sensor which corresponds to the sensor information, e.g., the temperature detected by the temperature sensor 111, is stored in the storage section 112 (S303).

Thereafter, it is determined whether or not the information association control section 113 has received a request of referring to the detected temperature from the microwave oven 201 (S304). When the information association control section 113 receives such a reference request, the detected temperature corresponding to the reference request is transmitted (S305). If the reference request has not been received at step S304, it is determined whether or not a predetermined time period has elapsed since the detected temperature was stored in the storage section 112 (S306). The above determinations of steps S304 and S306 are repeated till the predetermined time period elapses. If the predetermined time period has elapsed before a reference request is received from the microwave oven 201, the detected temperature stored in the storage section 112 is cleared (S307), and a series of the above processes are repeated. Further, along with the above processes, a response is made to the search conducted by the microwave oven 201 for an associated device (S310).

Meantime, at the microwave oven 201, it is determined whether or not a user has performed an operation at the operation section 216 as in the refrigerator 101 (S401). The above determination is repeated till any user operation is detected. When a user operation is detected, it is determined whether or not the user operation corresponds to a user operation that triggers the search for an associated device stored in the request information definition (S402). If not, the above determinations of steps S401 and S402 are repeated. Herein, the user operation that triggers the search for the associated device may be a user operation that actually requires a detection result of a sensor, e.g., the heating instruction operation, or the like. However, the trigger operation is preferably an operation which is performed prior to a user operation or device operation that actually requires a detection result of a sensor, e.g., opening of the door of the microwave oven 201. That is, the associated device is searched for in response to such a user operation, whereby a margin is given to a response time of the device. Furthermore, the search efficiency is improved as compared with embodiment 1 where the search is performed irrespective of the user's operation.

If the user's operation corresponds to a user operation that triggers the search for the associated device at step S402, it is determined whether or not previously-searched devices in an associated device list stored in the information association control section 212 includes an associated device having a required sensor (S403). If not, it is determined whether or not such a target device is connected to a network (S404). If an associated device is not found, the processes of step S401 and subsequent steps are repeated. If found, the found device is added to the associated device list (S406).

If the intended device has already been included in the associated device list at step S402 or is added to the list at step S406, it is determined whether or not a detection result of a sensor included in the device (herein, the compartment temperature of the refrigerator 101) has became necessary within a predetermined time period (S407). If there is a possibility that a detection result of the sensor of an associated device becomes necessary, for example, when the door of the microwave oven 201 is opened, associated devices are searched for to make a preparation for referring to the detection result of the sensor. However, if a user operation that actually requires the detection result of the sensor is not performed, nothing is done before the processes of step S401 and subsequent steps are repeated. Alternatively, if there is a possibility (or it is expected) that the detection result of the sensor becomes necessary, transmission of the detection result may be requested of the refrigerator 101 beforehand irrespective of whether or not it becomes actually necessary.

On the other hand, if the compartment temperature of the refrigerator 101 becomes necessary at step S407, a request for referring to the detection result of the sensor is transmitted to the refrigerator 101 (S408). If the compartment temperature is transmitted from the refrigerator 101 in response to the request, for example, a heating operation is performed with an optimum heating power based on the compartment temperature.

(Other Embodiments)

In another example of association of sensor information which will be described herein, sensor information obtained from a plurality of sensors provided to several places in an ordinary home are utilized to determine whether or not somebody is at home.

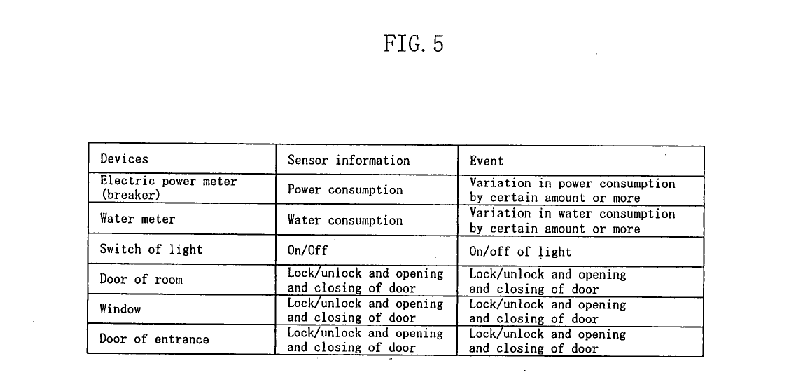

Various sensors are provided inside and outside a house as listed in FIG. 5. For example, a microcomputer incorporated in an entrance door requests detection results of the sensors, and the status of the house, e.g., whether or not somebody is at home, is determined based on the detection results.

Specifically, for example, when leaving home, electric appliances, such as the room light, air conditioner, and the like, are powered off, so that the power consumption detected by a electric power meter decreases. If it is detected under such a condition that the entrance door is opened and closed and the door is locked from outside, it is determined that nobody is at home.

When a user (somebody of the household) arrives at home, for example, it is first detected that the entrance door is unlocked from outside. Within a predetermined time period after it is detected, the user uses water to wash his/her hands so that the water consumption detected by a water meter increases. Alternatively, the light of the living room is turned on so that the power consumption increases. In view of such detection results of the sensors, it is determined that somebody of the household arrived at home. If such a series of detection results are not obtained, it is determined that some abnormality occurred. For example, a warning stating that “there is a possibility of an intruder”, or the like, is transmitted to a cellular mobile phone of a member of the household.

In the case of determining whether or not somebody is at home by using a plurality of sensors as described above, the efforts that are required in a commonly-employed security system, e.g., getting out of a room within a predetermined time period after a monitoring unit is activated, and deactivating the monitoring unit within a predetermined time after entering the room, are not necessary. Thus, the user can behave ordinarily and avoid a malfunction and burdensome troubles.

### INDUSTRIAL APPLICABILITY

As described above, according to the present invention, even a detection result for a detection target which is difficult to obtain with a single device is readily obtained by cooperation with a device which can obtain such a detection result, such that the operation control of the device is performed more appropriately, and various operation control are achieved.

1. A sensor information association device, comprising:
a sensor;
storage means for storing a history of a detection result detected by the sensor at a predetermined timing determined according to a user operation at the sensor information association device;
detection result request reception means for receiving from another device a request for the detection result detected by the sensor, the another device being capable of operating independently; and
detection result transmission means for transmitting the detection result detected at the predetermined timing determined according to the user operation and stored in the storage means to the another device according to the request.

2. (Cancelled)

3. The sensor information association device of claim 1, wherein:
the sensor information association device and the another device deal with a same material; and
the predetermined timing is a timing determined according to a user operation performed by a user for transferring the material to the sensor information association device.

4. (Cancelled)

5. (Cancelled)

6. (Cancelled)

7. A sensor information association device capable of operating independently, comprising:
detection result request means for requesting a detection result at a predetermined second timing determined according to the status of the sensor information association device, the detection result being detected by a sensor provided to another device at a first timing and stored;
detection result reception means for receiving the detection result transmitted in response to the request; and
wherein the second timing is a timing when a user performs a user operation which is expected to instruct the sensor information association device to perform operation control using the detection result obtained by the sensor.

8. (Cancelled)

9. (Cancelled)

10. (Cancelled)

11. (Cancelled)

12. (Cancelled)

13. (Cancelled)

14. (Cancelled)

15. (Cancelled)

16. (Cancelled)

17. The sensor information association device of claim 1, wherein:
the sensor information association device is a refrigerator;
the another device is a heating cooking appliance;
the sensor is a temperature sensor for detecting a compartment temperature of the refrigerator; and
the predetermined timing is a timing when the refrigerator is opened and closed.

18. (Cancelled)

19. The sensor information association device of claim 7, wherein:
the sensor information association device is a heating cooking appliance;
the another device is a refrigerator;
the sensor is a temperature sensor for detecting a compartment temperature of the refrigerator;
the first timing is a timing when the refrigerator is opened and closed; and
the heating cooking appliance performs heating control based on the received detection result.

20. The sensor information association device of claim 1, further comprising operation status detection means for detecting the status determined according to a user operation at the sensor information association device, wherein
when the operation status detection means detects that a predetermined user operation is performed, the storage means stores a history of the detection result detected by the sensor in conjunction with the predetermined user operation.

21. The sensor information association device of claim 20, wherein the detection result transmission means transmits information obtained by correcting the detection result of the sensor according to the operation status detected by the operation status detection means.

22. The sensor information association device of claim 21, wherein the detection result transmission means is capable of transmitting information that indicates the time elapsed after the status has been detected by the operation status detection means together with the detection result of the sensor.

23. The sensor information association device of claim 20, further comprising a provided sensor information database in which information indicating that the detection result of the sensor at the time of the user operation can be provided is registered in conjunction with a user operation detected by the operation status detection means, wherein
when the operation status detection means detects that a predetermined user operation is performed based on the provided sensor information database, the storage means stores a history of the detection result detected by the sensor based on the provided sensor information database.

24. The sensor information association device of claim 20, wherein:
the storage means stores a plurality of detection results according to the operation status and operation timing; and
the detection result transmission means selectively transmits any of the plurality of detection results according to a request from the another device which is issued based on the operation status of the another device or the detection result of the sensor provided to the another device.

25. The sensor information association device of claim 24, wherein:
the sensor information association device is a refrigerator;
the another device is a heating cooking appliance;
the sensor is a temperature sensor for detecting a compartment temperature of a freezer compartment and a vegetable compartment of the refrigerator;
the predetermined timing is a timing when the freezer compartment or vegetable compartment is opened and closed; and
the detection result transmission means transmits the compartment temperature of the freezer compartment or vegetable compartment, which is stored in the storage means, according to whether the operation status of the another device is a thawing instruction operation or heating instruction operation or according to a request based on the surface temperature of the material to be cooked.

26. The sensor information association device of claim 7, further comprising operation status detection means for detecting the status determined according to a user operation at the sensor information association device, wherein
when the operation status detection means detects that a predetermined user operation is performed, the detection result request means requests a detection result detected by the sensor of the another device and stored.

27. The sensor information association device of claim 26, further comprising a request sensor information database in which information indicating an data item detected by the sensor of the another device usable when the user operation is performed is registered in conjunction with a user operation detected by the operation status detection means, wherein
when the operation status detection means detects that a predetermined user operation is performed, the detection result transmission means requests a detection result detected by the sensor of the another device and stored, based on the request sensor information database.

28. The sensor information association device of claim 26, wherein when the operation status detection means detects that a predetermined user operation is performed, any of a plurality of detection results detected by the sensor of the another device and stored is used based on the performed user operation or a detection result of a sensor of the sensor information association device.

29. The sensor information association device of claim 28, wherein:
the sensor information association device is a heating cooking appliance;
the another device is a refrigerator; and
the compartment temperature of the freezer compartment or vegetable compartment of the refrigerator, which is detected by the sensor of the another device, is used based on whether the performed user operation is a thawing instruction operation or heating instruction operation or based on the surface temperature of the material to be cooked which is detected by the sensor of the sensor information association device.

30. The sensor information association device of claim 26, wherein a detection result obtained by the sensor of the another device is corrected according to the time interval that elapses between a time when detection is performed by the sensor of the another device and a time when the operation status detection means detects that the predetermined user operation has been performed, or according to a detection result of a sensor of the sensor information association device.

31. A sensor information association system comprising:
a sensor information provision device; and
a sensor information using device capable of operating independently, wherein
the sensor information provision device includes
a sensor,
detection result request reception means for receiving from the sensor information using device a request for the detection result detected by the sensor, and
detection result transmission means for transmitting the detection result detected at a first timing determined according to the user operation and stored in the storage means to the sensor information using device according to the request,
the sensor information using device includes
detection result request means for requesting a detection result of the sensor which is detected at the sensor information provision device, and
detection result reception means for receiving the detection result transmitted according to the request,
any one of the sensor information provision device and the sensor information using device includes storage means for storing a history of a detection result detected by the sensor at the predetermined first timing determined according to a user operation at the sensor information provision device, and
the sensor information using device uses the detection result stored in the storage means at a second timing when a user performs a user operation which is expected to instruct the sensor information using device to perform operation control using the detection result obtained by the sensor.

32. The sensor information association system of claim 31, wherein:
the sensor information provision device and the sensor information using device further include first and second operation status detection means, respectively, each of which detects the status corresponding to a performed user operation;
when the first operation status detection means detects that a predetermined user operation is performed at the sensor information provision means, the storage means of the sensor information provision means stores a plurality of detection results detected by the sensor in conjunction of the predetermined user operation and operation timing; and
any of the plurality of detection results stored in the storage means of the sensor information provision means is selectively used based on an user operation performed at the sensor information using device, which is detected by the second operation status detection means, or based on a detection result of a sensor of the sensor information using device.
